(19) United States
(12) Patent Application Publication
(10) Pub. No.: US 2022/0273689 A1
(43) Pub. Date: Sep. 1, 2022

(54) POTENTIATION OF ANTIVIRAL NUCLEOBASES AS RNA VIRUS THERAPY

(71) Applicant: REGENTS OF THE UNIVERSITY OF MINNESOTA, Minneapolis, MN (US)

(72) Inventors: Laurent Francois BONNAC, Minneapolis, MN (US); Robert James GERAGHTY, Minneapolis, MN (US)

(73) Assignee: REGENTS OF THE UNIVERSITY OF MINNESOTA, Minneapolis, MN (US)

(21) Appl. No.: 17/625,020

(22) PCT Filed: Jul. 8, 2020
(86) PCT No.: PCT/US2020/041133

§ 371 Date: Jan. 5, 2022

Related U.S. Application Data
(60) Provisional application No. 62/872,071, filed on Jul. 9, 2019.

(51) Int. Cl. A61K 31/7076; A61P 31/14; A61K 31/4965; A61K 31/706; A61K 31/4196; A61K 31/7056; A61K 31/4164; A61K 31/52; A61K 31/365

(52) U.S. Cl. CPC A61K 31/7076; A61P 31/14; A61K 31/4965; A61K 31/706; A61K 31/4196; A61K 31/7056; A61K 31/4164; A61K 31/52; A61K 31/365

(57) ABSTRACT

Described herein are methods of treating RNA virus infections with a therapeutic combination of an antiviral nucleobase compound and a de novo nucleotide biosynthesis inhibitor (DNNBi). An aspect of the invention is a method for the treatment of an RNA virus infection comprising administering a therapeutic combination as a combined formulation or by alternation to a patient, wherein the therapeutic combination comprises therapeutically effective amounts of (i) an antiviral nucleobase or a pharmaceutically acceptable salt thereof, and (ii) a de novo nucleotide biosynthesis inhibitor (DNNBi) or a pharmaceutically acceptable salt thereof.

### CROSS-REFERENCE TO RELATED APPLICATIONS

This application claims priority to U.S. Provisional Patent Application No. 62/872,071, filed on 9 Jul. 2019, which is incorporated by reference in its entirety.

### FIELD OF THE INVENTION

The invention relates generally to treatment of viral infection in patients by administering the combination of an antiviral nucleobase and a potentiator compound.

### BACKGROUND OF THE INVENTION

Dengue, zika, influenza, SARS-cov-2 and other emerging viruses infect hundreds of millions of people each year and represent a global health emergency requiring the development of potent antiviral treatments. Dengue virus (DENV) is a worldwide health threat, with hundreds of millions of people infected yearly in more than 100 countries (S. Bhatt et al., (2013) Nature 496, 504-507). There are four known DENV serotypes. A first infection with one serotype followed by a second infection with another serotype may result in severe disease (S. B. Halstead, (2003) Adv Virus Res 60:421-467; C. P. Simmons, (2015) New Engl. Jour. of Med. 373:1263-1264). For these and other issues, vaccines designed for pan-serotype protection, including the commercial dengue vaccine approved and used in a few countries, have yielded mixed results (S. R. Hadinegoro et al. (2015) New Engl. Jour. of Med. 373:1195-1206). Safety and partial efficacy concerns in addition to cost, storage and delivery issues may hinder implementation of vaccines in many countries.

Zika virus (ZIKV), like DENV, is a member of the Flaviviridae family of viruses and both are spread to humans by mosquitos. ZIKV is likely spread by additional routes including intimate contact and mother-to-fetus during pregnancy (L. R. Petersen et al (2016) N Engl. Jour. Med 374:1552-1563). Although isolated in the late 1940's, ZIKV research has not been a high priority in the U.S. because infection was often believed to be asymptomatic or mild with short-lived symptoms. ZIKV has become a high priority due to the dramatic rise in the number of cases, geographic spread of the outbreak, and ZIKV infection during the first trimester of pregnancy increases the risk of fetal microcephaly and other central nervous system anomalies (M. A. Johansson et al (2016) N Engl. Jour. Med, 375(1):1-4). There are serious concerns about ZIKV establishing itself in areas of high population density as DENV has done. The development of anti-ZIKV drugs is an effective way to control ZIKV outbreaks and spread. Effective ZIKV drugs could be used prophylactically to prevent infections and as treatment to reduce viral load, thereby reducing virus spread. There are currently no approved drugs to treat DENV or ZIKV infection. Thus far, classical antiviral approaches (e.g. NS5 polymerase inhibitors, entry inhibitors, protease inhibitors, etc.) have yet to provide treatments for DENV infection and therefore the investigation of new antiviral strategies is warranted (Y. L. Chen, et al (2015) Antiviral Res 122:12-19; S. P. Lim et al in Antiviral Res. (2013 Elsevier B. V, Netherlands, 2013), vol. 100, pp. 500-519; J. G. Low, et al (2017) J. Infect. Dis. 215:S96-S102).

Influenza A virus (IAV) is an orthomyxovirus with a segmented single-stranded RNA genome and lipid enveloped viral particles. IAV is a significant cause of morbidity and mortality worldwide despite vaccine and antiviral availability. The CDC estimates between 290,000-640,000 deaths worldwide associated with IAV infections (Iuliano, et al, The Lancet, 391:1285-1300. 2018). The currently used IAV vaccines suffer from several challenges including inefficient production, requirement for annual inoculation and the need for modification for use in the elderly population (House and Subbarao, Influenza Vaccines: Challenges and Solutions, Cell Host Microbe. 2015. 17(3):295-300). Currently, three classes of inhibitors are approved for treatment of IAV infections, neuraminidase inhibitors (NAIs), M2 ion channel inhibitors (M2Is) and PA mRNA cap cleavage inhibitors (CCIs). Rampant resistance to the M2Is amantadine and rimantadine in circulating IAV strains has rendered those drugs nearly useless (Hsu et al, Ann. Intern. Med. 2012, 156, 512-524). The NAIs, including zanamivir and oseltamivir approved in the U. S. as well as peramivir and laninamivir approved in other countries, must be used judiciously to avoid generating wide-spread resistance. Baloxavir marboxyl is a recently been approved CCI and with results as good or better than the NAIs but resistance to it is present in circulating IAV strains (NEJM vol 379(10):913-923. 2018). Baloxavir overuse could increase the frequency of resistant IAV and render the drug ineffective similarly to the M2Is.

Coronaviruses have large (26-32 kb) plus-sense single-strand RNA genomes and enveloped viral particles with distinctive lollipop spike proteins projecting from their surface. Disease causing human coronaviruses identified thus far are mainly from the Alphacoronavirus and Betacoronavirus genera. Human alphacoronaviruses NL-63 and 229E, as well as human betacoronaviruses HKU1 and OC43 (HCoV-OC43), cause cold-like symptoms and the conditions are usually non-life threatening. Betacoronaviruses that normally circulate in animals but sporadically infect humans can cause severe disease. In 2002 severe acute respiratory syndrome (SARS)-CoV was identified to cause more than 800 deaths and since 2012 Middle East respiratory syndrome (MERS) CoV has caused more than 800 fatalities (WHO). In late 2019 a new human betacoronavirus emerged, SARS-CoV-2, that is related but genetically distinct from SARS-CoV and MERS. SARS-CoV-2 causes coronavirus disease 2019 (COVID-19), an escalating pandemic infecting millions of people worldwide (Zhu, N. et al (2020) N Engl. J. Med. 382:727-733; Coronaviridae Study Group of the International Committee on Taxonomy of Viruses. (2020) Nat. Microbiol. 5:536-544). Symptoms of COVID-19 infection include respiratory illness, sometimes severe requiring hospitalization and breathing assistance (Wolfel, R., et al (2020) Nature 581:465-469). There are no antivirals for treatment. In addition, based upon experience treating other RNA viruses, multiple drugs will be required to prevent resistance development.

Although nucleoside analogues represent a successful class of antiviral drugs, the discovery of new antiviral nucleosides has been impaired by several hurdles including the toxicity of the potential drugs as well as synthetic challenges. In addition, potentially antiviral nucleosides frequently suffer from poor metabolic conversion to the active triphosphate form required by the viral polymerase. The first phosphorylation of the nucleoside analogue is often the rate limiting step to obtain the active nucleoside triphosphate used by the viral polymerase (A. R. Van Rompay, et al (2000) Pharmacol. Ther. 87:189-198; A. R. Van Rompay, et al (2003) Pharmacol. Ther. 100:119-139).

The anti-viral drug favipiravir, (AVIGAN®, ABIGAN®, FABIFLU®) approved in Japan to treat influenza, possesses a rare and remarkably broad antiviral activity against many viruses, including DENV and ZIKV, without associated toxicity in cell culture systems (U.S. Pat. Nos. 6,787,544; 8,759,354; U.S. RE43748). Favipiravir exerts an antiviral effect as a nucleotide analog through a combination of chain termination, slowed RNA synthesis and lethal mutagenesis (Shannon A., et al (2020) bioRxiv, 1-19, https://doi.org/10.1101/2020.05.15.098731). However, outside of influenza A virus infections, favipiravir is not effective in vivo. For example, the JIKI trial evaluating the nucleobase T-705/favipiravir against Ebola virus demonstrated efficacy (PLoS Med. 2016; 13(3):e1001967. Epub 2016 Mar. 2. doi: 10.1371/journal.pmed.1001967. PubMed PMID: 26930627; PMCID: PMC4773183) yet suboptimal drug concentration in patients (PLoS Negl Trop Dis. 2017; 11(2):e0005389. Epub 2017 Feb. 24. doi: 10.1371/journal.pntd.0005389. PubMed PMID: 28231247; PMCID: PMC5340401) highlighting the need of strategies to enhance favipiravir bioactivation and antiviral activity.

Severe acute respiratory syndrome coronavirus 2 (SARS-CoV-2) is the strain of coronavirus that causes coronavirus disease 2019 (COVID-19), an escalating pandemic infecting millions of people worldwide (Zhu, N. et al (2020) N. Engl. J. Med. 382:727-733; Coronaviridae Study Group of the International Committee on Taxonomy of Viruses. (2020) Nat. Microbiol. 5:536-544). Symptoms of COVID-19 infection include respiratory illness, sometimes severe requiring hospitalization and breathing assistance (Wolfel, R., et al (2020) Nature 581:465-469). SARS-CoV-2 is a single-stranded RNA virus with a genome of approximately 30 kB encoding 29 proteins (Vijgen L., et al (2020) J. Virol. 79(3):1595-1604).

With the increasing threats of emerging and reemerging viruses, there is a need for broad spectrum antivirals to treat RNA virus infections, such as COVID-19 (Wang, M. et al (2020) Cell Research 30(3):269-271; Li, G. and De Clercq, E. (2020) Nature Reviews Drug Discovery 19(3):149-150; Dong, L. et al (2020) Drug discoveries & therapeutics 14(1):58-60; Liu, C. et al (2020) ACS Central Science 6(3):315-331; Cai, Q. et al (2020) Engineering, https://doi.org/10.1016/j.eng.2020.03.007). New combination therapies that provide an enhanced therapeutic safety and efficacy, yield lower resistance and predict higher patient compliance are needed. In particular, strategies to increase favipiravir efficacy as a first-line emerging virus treatment are therefore needed.

### DETAILED DESCRIPTION

An aspect of the invention is a method for the treatment of an RNA virus infection comprising administering a therapeutic combination as a combined formulation or by alternation to a patient, wherein the therapeutic combination comprises therapeutically effective amounts of (i) an antiviral nucleobase or a pharmaceutically acceptable salt thereof; and (ii) a de novo nucleotide biosynthesis inhibitor (DNNBi) or a pharmaceutically acceptable salt thereof.

In one embodiment, the viral infection is selected from dengue virus (DENV), Zika virus (ZIKV), Ebola virus, West Nile virus. severe acute respiratory syndrome (SARS) virus, Middle East Respiratory syndrome (MERS) coronavirus, rabies virus, common cold viruses, influenza, hepatitis C, West Nile fever, polio. measles, respiratory syncytial virus, Nipah virus, Lassa fever virus, and SARS-CoV-2.

In one embodiment, the antiviral nucleobase is selected from 1H-1,2,4-triazole-3-carboxamide, 5-hydroxy-1H-imidazole-4-carboxamide, 3-hydroxypyrazine-2-carboxamide, 9H-purine-2,6-diamine; and 6-fluoro-3-hydroxypyrazine-2-carboxamide.

In one embodiment, the DNNBi is (2R,3S,4R,5R)-2-(hydroxymethyl)-5-(6-(methylthio)-9H-purin-9-yl)tetrahydrofuran-3,4-diol (6-MMPR).

In one embodiment, the DNNBi is a pro-drug of 6-MMPR.

In one embodiment, the antiviral nucleobase is favipiravir and the DNNBi is 6-MMPR.

In one embodiment, the antiviral nucleobase is 3-hydroxypyrazine-2-carboxamide (T-1105) and the DNNBi is 6-MMPR.

In one embodiment, the therapeutic combination is administered to the patient as a combined formulation.

In one embodiment, the therapeutic combination is a solid, oral dosage form.

In one embodiment, the solid, oral dosage form is a tablet or capsule.

In one embodiment, the therapeutic combination is administered to the patient by alternation during a dosing regimen.

Another aspect of the invention is a method of selecting for patients likely to respond to a combination of an antiviral nucleobase and a de novo nucleotide biosynthesis inhibitor (DNNBi), wherein the patient has been previously treated with a viral RNA polymerase inhibitor.

In one embodiment, a biological sample from the patient has been characterized to contain an NS1 viral protein, viral RNA or the presence of viral antibodies within 7 days after onset of symptoms associated with an RNA viral infection.

Another aspect of the invention is a method of inhibiting replication of a virus comprising treating a virus-infected cell with an antiviral nucleobase and a de novo nucleotide biosynthesis inhibitor (DNNBi).

Another aspect of the invention is a pharmaceutical composition comprising therapeutically effective amounts of an antiviral nucleobase and a potentiating, de novo nucleotide biosynthesis inhibitor (DNNBi), and an excipient.

In one embodiment, the pharmaceutical composition comprises favipiravir and 6-MMPR.

In one embodiment, the pharmaceutical composition is in solid, oral dosage form.

In one embodiment of the pharmaceutical composition, the solid, oral dosage form is a tablet or capsule.

In one embodiment of the pharmaceutical composition, the antiviral nucleobase and (DNNBi) are in synergistic amounts.

In one embodiment of the pharmaceutical composition, the antiviral nucleobase is favipiravir and the DNNBi is 6-MMPR.

In one embodiment of the pharmaceutical composition, the antiviral nucleobase is 3-hydroxypyrazine-2-carboxamide (T-1105) and the DNNBi is 6-MMPR.

The invention includes all reasonable combinations and permutations of the embodiments.

### BRIEF DESCRIPTION OF THE DRAWINGS

FIG. 1 shows activation pathways of antiviral nucleobases and nucleosides.

FIG. 2 shows inhibition of endogenous de novo nucleotides synthesis potentiates the antiviral properties of antiviral nucleobases.

FIG. 3A shows potentiation of antiviral activity for ribavirin (Rib) nucleobase with 6-methyl-mercaptopurine riboside (6-MMPR). All concentrations in μM (micromolar). MPA=mycophenolic acid control inhibitor (1 μM)

FIG. 3B shows potentiation of antiviral activity for mizoribine (Miz) nucleobase with 6-MMPR. All concentrations in μM. MPA=mycophenolic acid control inhibitor (1 μM)

FIG. 4A shows nucleobase and DNNBi combinations produce a synergistic effect for reduction in DENV replicon replication. DENV replicon cells in 96-well plates were treated with the indicated concentrations of the two compounds and analyzed for luciferase activity. Samples were prepared in triplicate and experiments performed two independent times. T-705=5a, and AM28=6-MMPR. Analysis of results using MacSynergy II software to explore possible synergy.

FIG. 4B shows nucleobase and DNNBi combinations produce a synergistic effect for reduction in DENV replicon replication. DENV replicon cells in 96-well plates were treated with the indicated concentrations of the two compounds and analyzed for luciferase activity. Samples were prepared in triplicate and experiments performed two independent times. T-1105=3a, and AM28=6-MMPR. Analysis of results using MacSynergy II software to explore possible synergy.

FIG. 4C shows nucleoside and DNNBi combinations produce a synergistic effect for reduction in DENV replicon replication. DENV replicon cells in 96-well plates were treated with the indicated concentrations of the two compounds and analyzed for luciferase activity. Samples were prepared in triplicate and experiments performed two independent times. T-1106=3b and AM28=6-MMPR Analysis of results using MacSynergy II software to explore possible synergy.

FIG. 5A shows viability results for treatment of DENV replicon cells with nucleobase T-1105 (3a) and DNNBi AM28 (6-MMPR).

FIG. 5B shows viability results for treatment of DENV replicon cells with nucleobase T-705 (5a) and DNNBi AM28 (6-MMPR).

FIG. 5C shows viability results for treatment of DENV replicon cells with nucleoside T-1106 (3b) and DNNBi AM28 (6-MMPR).

FIG. 6A shows nucleobase and DNNBi combinations produce a synergistic effect for reduction in DENV replication in a three-dimensional plot. Huh-7 cells were inoculated with DENV and treated with nucleobase T-1105 (3a) and AM28 (6-MMPR), nucleobase plus DMSO or AM28 plus DMSO. Infectious virus produced by treated cells was titered and results analyzed by MacSynergy II program. ND=not detected

FIG. 6B shows nucleobase T-1105 (3a) and DNNBi combinations produce a synergistic effect for reduction in DENV replication in a bar plot of nucleobase and DNNBi concentrations. Huh-7 cells were inoculated with DENV and treated with nucleobase and AM28 (6-MMPR), nucleobase plus DMSO or AM28 plus DMSO. Infectious virus produced by treated cells was titered and results analyzed by MacSynergy II program. ND=not detected

FIG. 7A shows cell viability results for treatment of Huh-7 cells with nucleobase T-1105=3a.

FIG. 7B shows cell viability results for treatment of Huh-7 cells with AM28=6-MMPR.

FIG. 8A shows nucleobase and DNNBi combination produces a synergistic effect for reduction in ZIKV replication in a three-dimensional plot. Huh7 cells were inoculated with ZIKV and treated with different concentrations of T-1105=3a nucleobase and 0.1 μM AM28 (6-MMPR) or DMSO. Infectious virus produced by treated was titered and results analyzed by MacSynergy II software.

FIG. 8B shows nucleoside and DNNBi combination does not produce a synergistic effect for reduction in ZIKV replication in a three-dimensional plot. Huh7 cells were inoculated with ZIKV and treated with different concentrations of T-1106=3b nucleoside and 0.1 μM AM28 (6-MMPR) or DMSO. Infectious virus produced by treated was titered and results analyzed by MacSynergy II software.

FIG. 8C shows nucleobase and DNNBi combination produces a synergistic effect for reduction in ZIKV replication in a three-dimensional plot. Huh7 cells were inoculated with ZIKV and treated with different concentrations of T-705=5a favipiravir nucleobase and 0.1 μM AM28 (6-MMPR) or DMSO. Infectious virus produced by treated was titered and results analyzed by MacSynergy II software.

FIG. 9A shows nucleobase and DNNBi combination produces a synergistic effect for reduction in DENV replication in a three-dimensional plot. Huh7 cells were inoculated with DENV and treated with different concentrations of T-1105=3a nucleobase and 0.1 μM AM28 (6-MMPR) or DMSO. Infectious virus produced by treated was titered and results analyzed by MacSynergy II software.

FIG. 9B shows nucleoside and DNNBi combination does not produce a synergistic effect for reduction in DENV replication in a three-dimensional plot. Huh7 cells were inoculated with DENV and treated with different concentrations of T-1106=3b nucleoside and 0.1 μM AM28 (6-MMPR) or DMSO. Infectious virus produced by treated was titered and results analyzed by MacSynergy II software.

FIG. 9C shows nucleobase and DNNBi combination produces a synergistic effect for reduction in DENV replication in a three-dimensional plot. Huh7 cells were inoculated with DENV and treated with different concentrations of T-705=5a favipiravir nucleobase and 0.1 μM AM28 (6-MMPR) or DMSO. Infectious virus produced by treated was titered and results analyzed by MacSynergy II software.

FIG. 10A shows a plot of results of a Titer Reduction assay where 6-MMPR (6-methylmercaptopurine riboside) synergistically increases the ability of favipiravir (T705) to inhibit the production of infectious coronavirus particles.

FIG. 10B shows a three dimensional plot of synergy analysis by MacSynergy with varying concentrations of favipiravir (T705) and 6-MMPR. The dome indicates a clear synergistic effect.

FIG. 11A shows a protection of human cells from IAV-induced cell death when favipiravir (T-705) is combined with 6MMPr.

FIG. 11B shows the synergistic effect of a clear synergy peak over the additive plane in the 3-D MacSynergy plot.

FIG. 12 shows a flow chart of the synergistic potentiation mechanism of treatment of an infected cell with antiviral-nucleobases by de novo nucleotide biosynthesis inhibitors.

FIG. 13A shows a plot of viral RNA copies per ml serum from a DENV mouse model. The mice were treated with Vehicle, the combination T-1105 (3a)+6-MMPR, 1105 only, and PBS. Viral genomes were detected and quantitated by RT-qPCR at 2 or 4 days post infection.

FIG. 13B shows a plot of body weight from mice during the study of FIG. 13A where the mean plus error for percent original body weight for 11 days post-infection.

### DEFINITIONS

Unless defined otherwise, technical and scientific terms used herein have the same meaning as commonly understood by one of ordinary skill in the art to which this invention belongs.

The words “comprise,” “comprising,” “include,” “including,” and “includes” when used in this specification and claims are intended to specify the presence of stated features, integers, components, or steps, but they do not preclude the presence or addition of one or more other features, integers, components, steps, or groups thereof.

The terms “treat” and “treatment” refer to both therapeutic treatment and prophylactic or preventative measures, wherein the object is to prevent or slow down (lessen) an undesired physiological change or disorder, as a consequence of the onset, progression, or transmission of a viral infection. For purposes of this invention, beneficial or desired clinical results include, but are not limited to, alleviation of symptoms, diminishment of extent of infection, stabilized (i.e., not worsening) state of infection, delay or slowing of infection progression, amelioration or palliation of the infected state, and reoccurrence (whether partial or total), whether detectable or undetectable. “Treatment” can also mean prolonging survival as compared to expected survival if not receiving treatment. Those in need of treatment include those already with the condition or related disorders as well as those prone to have the condition or disorder or those in which the condition or disorder is to be prevented.

The phrase “therapeutically effective amount” means an amount of a compound of the present invention that (i) treats the particular infection, (ii) attenuates, ameliorates, or eliminates one or more symptoms of the particular infection, or (iii) prevents or delays the onset of one or more symptoms of the particular infection described herein. Furthermore, “therapeutically effective amount” is an amount of a drug that is low enough to be non-toxic, yet sufficient to achieve a therapeutic result, including eliminating, reducing, and/or slowing the progression of a condition or symptom thereof. The therapeutically effective amount may depend on biological factors. Achieving a therapeutic result can be measured by physician or other qualified medical personnel using objective evaluations known in the art, or it can be measured by individual, subjective patient assessment.

The term “detection” includes any means of detecting, including direct and indirect detection.

The term “prognosis” is used herein to refer to the prediction of the likelihood of infection-attributable death or progression, including, for example, recurrence, spread, drug resistance, and transmission of the infection.

The term “prediction” (and variations such as predicting) is used herein to refer to the likelihood that a patient will respond either favorably or unfavorably to a drug or a combination therapy regimen. In one embodiment, the prediction relates to the extent of those responses. In another embodiment, the prediction relates to whether and/or the probability that a patient will survive following treatment, for example treatment with a particular therapeutic agent and/or other treatment options. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, or whether long-term survival of the patient, following a therapeutic regimen is likely.

The term “increased resistance” to a particular therapeutic agent or treatment option, when used in accordance with the invention, means decreased response to a standard dose of the drug or to a standard treatment protocol.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

The desired pharmaceutically acceptable salt may be prepared by any suitable method available in the art. For example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. Acids which are generally considered suitable for the formation of pharmaceutically useful or acceptable salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1 19; P. Gould, International J. of Pharmaceutics (1986) 33 201 217; Anderson et al, The Practice of Medicinal Chemistry (1996), Academic Press, New York; Remington's Pharmaceutical Sciences, 18^{th} ed., (1995) Mack Publishing Co., Easton Pa.; and in The Orange Book (Food & Drug Administration, Washington, D.C. on their website). These disclosures are incorporated herein by reference thereto.

The phrase “pharmaceutically acceptable” indicates that the substance or composition must be compatible chemically and/or toxicologically, with the other ingredients comprising a formulation, and/or the mammal being treated therewith.

The terms “synergistic” and “synergy” as used herein refer to a therapeutic combination which is more effective than the additive effects of the two or more single agents. A determination of a synergistic interaction between a compound of an antiviral nucleobase, or a pharmaceutically acceptable salt thereof, and one or more DNNBi potentiator agent may be based on the results obtained from the assays described herein. The results of these assays can be analyzed using the Chou and Talalay combination method and Dose-Effect Analysis with CalcuSyn® software in order to obtain a Combination Index (Chou and Talalay, 1984, Adv. Enzyme Regul. 22:27-55). The combinations provided by this invention have been evaluated in several assay systems, and the data can be analyzed utilizing a standard program for quantifying synergism, additivism, and antagonism among anticancer agents described by Chou and Talalay, in “New Avenues in Developmental Cancer Chemotherapy,” Academic Press, 1987, Chapter 2. Combination Index values less than 0.8 indicates synergy, values greater than 1.2 indicate antagonism and values between 0.8 and 1.2 indicate additive effects. The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes or in separate pills or tablets. In general, during alternation therapy, an effective dosage of each active ingredient is administered sequentially, i.e., serially, whereas in combination therapy, effective dosages of two or more active ingredients are administered together. Combination effects may also be evaluated using both the BLISS independence model and the highest single agent (HSA) model (Lehár et al. 2007, Molecular Systems Biology 3:80). BLISS scores quantify degree of potentiation from single agents and a BLISS score >0 suggests greater than simple additivity. An HSA score >0 suggests a combination effect greater than the maximum of the single agent responses at corresponding concentrations. Three dimensional synergistic analyses can also be processed using MacSynergy II software (Prichard, M. N. et al (1990) Antiviral Res 14:181-205; Smee, D. F. and Prichard, M. N. (2017) Antiviral Research 145:1-5).

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons. It is intended that all tautomeric forms of the compounds described herein are included as part of the present invention.

“Alkyl” refers to a straight or branched, saturated, aliphatic radical having the number of carbon atoms indicated. Alkyl can include any number of carbons. For example, C₁-C₄ alkyl includes, but is not limited to, methyl, ethyl, propyl, isopropyl, butyl, isobutyl, sec-butyl, and tert-butyl. Alkyl can also refer to alkyl groups having up to 30 carbons atoms, such as, but not limited to heptyl, octyl, nonyl, decyl, etc. Alkyl groups can be substituted or unsubstituted. “Substituted alkyl” groups can be substituted with one or more groups selected from halo, hydroxy, amino, oxo (═O), alkylamino, amido, acyl, nitro, cyano, and alkoxy.

“Heteroaryl” refer to a circular structure where one or more carbon atoms are optionally and independently replaced with one or more heteroatoms selected from N, O, and S. “Heteroaryl,” by itself or as part of another substituent, refers to a monocyclic or fused bicyclic or tricyclic aromatic ring assembly containing 5 to 16 ring atoms, where from 1 to 5 of the ring atoms are a heteroatom such as N, O or S. Heteroaryls can be 5-membered rings, 6-membered rings, and so on. Additional heteroatoms can also be present, including, but not limited to, B, Al, Si and P. The heteroatoms can be oxidized to form moieties such as, but not limited to, —S(O)— and —S(O)₂—. Exemplary heteroaryl groups include pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted. “Substituted heteroaryl” groups can be substituted with one or more groups selected from halo, hydroxy, amino, oxo (═O), alkylamino, amido, acyl, nitro, cyano, and alkoxy.

A “solid oral dosage form” refers to a formulation that is ready for administration to a subject via an oral route. Exemplary oral dosage forms include, but are not limited to, tablets, minitablets, capsules, caplets, powders, pellets, beads, granules, and pelletized tablets containing polymer-coated pellets. A dosage form can be a “unit dosage form,” which is intended to deliver one therapeutic dose per administration.

The term “excipient” refers to a substance formulated with an active pharmaceutical ingredient (API) of a therapeutic medication, included for the purpose of long-term stabilization, bulking up solid formulations that contain potent active ingredients in small amounts, or to confer a therapeutic enhancement on the active ingredient in the final dosage form, such as facilitating drug absorption, reducing viscosity, or enhancing solubility. Excipients can also be useful in the manufacturing process, to aid in the handling of the active substance concerned such as by facilitating powder flowability or non-stick properties, in addition to aiding in vitro stability such as prevention of denaturation or aggregation over the expected shelf life. The selection of appropriate excipients also depends upon the route of administration and the dosage form, as well as the active ingredient and other factors. In some formulations, excipients can be a key determinant of dosage form performance, with effects on pharmacodynamics and pharmacokinetics. Types of excipients for oral dosage formulations include antiadherents, binders, coatings, colors, disintegrants, flavors, glidants, lubricants, preservatives, sorbents, sweeteners, and vehicles.

### Antiviral Nucleobase Compounds

In order to overcome the potential first phosphorylation difficulty of nucleosides, nucleobases, the base of a nucleoside without its ribose moiety, are employed in the compositions of the invention. Enzyme mediated condensation of nucleobases with 5-phosphoribosyl-1-pyrophosphate (PRPP) to give the corresponding nucleoside-5′-monophosphate provide an alternative pathway to the antiviral nucleotide triphosphate active form. Thus, for a nucleoside where the first phosphorylation is inefficient, using its corresponding nucleobase could allow metabolic conversion to the corresponding nucleoside triphosphates thereby providing a more efficient metabolic conversion to the triphosphate (K. Negishi, et al (1994) Mutat. Res. 318:227-238) as demonstrated for nucleobases 5-fluorouracil and favipiravir (T-705) nucleobases (R. Agudo, et al (2009) Future Med Chem 1:529-539; S. Sierra, M et al (2000) J. Virol. 74:8316-8323; T. Baranovich et al., et al (2013) J. Virol. 87:3741-3751; A. Arias, et al (2014) Elife 3, e03679). However, in the context of targeting viruses that affect developing countries, nucleobases present key advantages over nucleosides. In addition to their different metabolic activation pathways, nucleobase analogues are considerably cheaper, more diverse and commercially available in higher numbers compared to corresponding nucleoside analogues. The chemical synthesis of a nucleobase is faster and simpler than the synthesis of the corresponding nucleoside. Similar to nucleosides, nucleobases possess their own cellular transporters (H. de Koning, et al (2000) Nucleobase transporters (review). Mol Membr Biol 17:75-94; D. A. Griffith, et al (1996) Biochim Biophys Acta 1286:153-181).

The nucleobase favipiravir, recently approved in Japan against influenza, possesses a broad antiviral activity. In late 2014, favipiravir was evaluated in the JIKI trial for Ebola virus infected patients and demonstrated moderate benefits by reducing the mortality rate for patients in the early infection stage of the disease (D. Sissoko et al (2016) PloS Med 13, e1001967; T. H. Nguyen et al (2017) PloS Negl Trop Dis 11, e0005389). Additional studies revealed that the administered dose during the JIKI trial failed to achieve the expected plasma concentration necessary to obtain an optimal antiviral effect.

The antiviral nucleobases of the invention are ambiguous base-pairing nucleobases. An ambiguous base-pairing nucleobase and corresponding ambiguous base-pairing nucleoside resemble more than one natural nucleoside due to structural variability. The ambiguous base-pairing nature of nucleobases and their structural variability may be due to (i) ionization (ii) tautomerism, (iii) bond rotation and/or (iv) ring opening which make ambiguous base-pairing compounds resemble more than one natural nucleotide. Ribavirin and T-705 nucleotide are embodiments of ambiguous base-pairing through bond rotation with the possible orientation of the amido group of the base in two different positions to either resemble adenosine or guanosine resulting in the antiviral effect.

Ambiguous base-pairing nucleobases may include electron-withdrawing groups such as halogen, cyano, nitro and amido, or electron-donating groups such as alkyl, alkene, and alkyne which alter the ionization state or tautomerism of pyrimidine or purine resulting in ambiguous base-pairing. Ambiguous base-pairing nucleobases may include rotatable amide groups that alter base-pairing, or modifications at position 5 of pyrimidines and position 7 of purines with groups such as alkyl, heterocycle, and heteroaryl which increase the stacking abilities of the nucleobase analogue during viral RNA synthesis yet decrease the specificity of the base-pairing. Ambiguous base-pairing nucleobases may include T-705 analogues, ribavirin nucleobase analogues, and sulfur containing nucleobases as depicted below. Desulfurization can occur, resulting in switching of base-pairing capacities.

Embodiments of antiviral nucleobases include pyrimidine analogs having the structures:

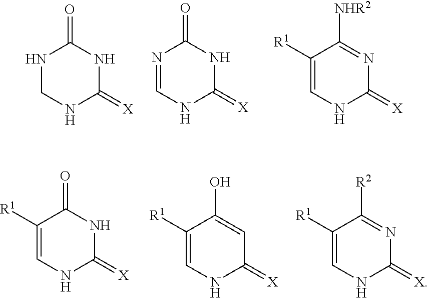

Embodiments of antiviral nucleobases include purine analogs having the structures:

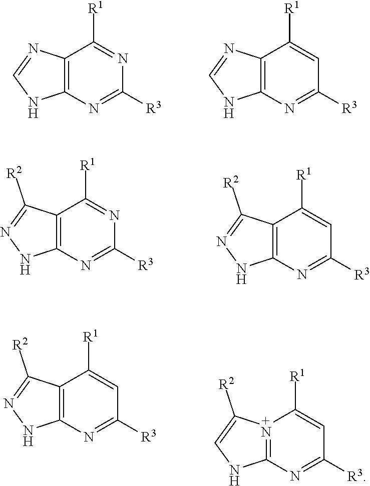

Embodiments of antiviral nucleobases include T-705 analogs having the structure:

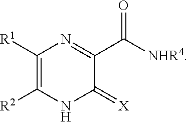

Embodiments of antiviral nucleobases include ribavirin analogs having the structure:

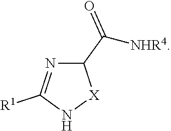

Substituents of the embodiments of antiviral nucleobases include wherein:

R¹ is selected from the group consisting of H, Me, F, Cl, Br, I, OH, NH₂, SH, OMe, NO₂, NHOH, NHOMe, NHNH₂, C═ONH₂, C₁-C₈ alkyl, and 5- or 6-membered heteroaryl;

R² is selected from the group consisting of H, OH, OMe, NH₂, NHMe, C═ONH₂, C₁-C₈ alkyl, and 5- or 6-membered heteroaryl;

R³ is selected from the group consisting of H, F, Cl, Br, I, OH, S, NH₂, SH, OMe, NO₂, NHOH, NHOMe, NHNH₂, C═ONH₂, C₁-C₈ alkyl, and 5- or 6-membered heteroaryl;

R⁴ is selected from the group consisting of H, NH₂ and C₁-C₈ alkyl; and

X is NR²,O or S.

### De Novo Nucleotide Biosynthesis Inhbitors (DNNBi)

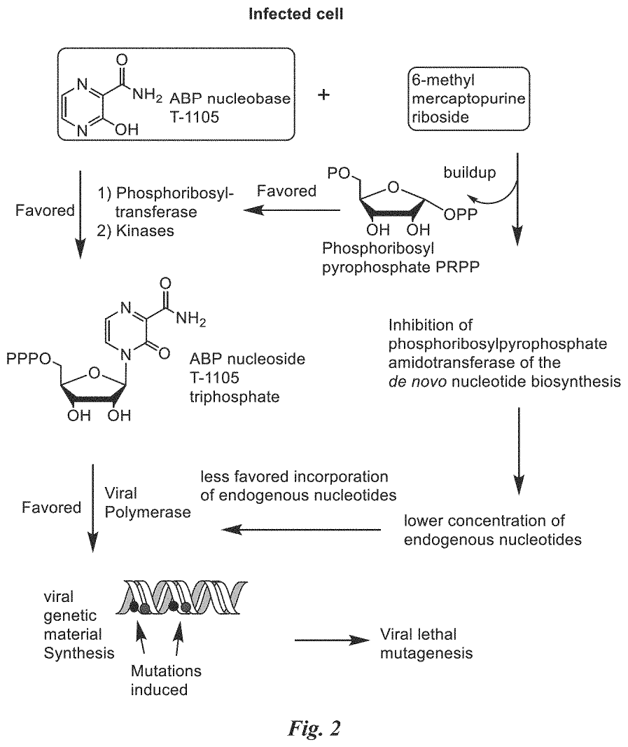

Nucleobases are paired with de novo nucleotide biosynthesis inhibitors (DNNBi) in combination therapy and combination formulations against viruses, including DENV, ZIKV, IAV (influenza A virus) and HCoV. 6-Methyl-mercaptopurine riboside (6-MMPR), and prodrugs thereof, is an embodiment of a DNNBi and is a strong potentiator of nucleobase anti-replicon activities (Example 1 and FIG. 2).

The de novo nucleotide biosynthesis (DNNB) is divided into two pathways, the DNNB of purine nucleotides (A and G) and the DNNB of pyrimidine nucleotides (U/T, C). Known inhibitors of these DNNB pathways will potentiate antiviral nucleobase activities. Examples of a range of inhibitors of the de novo purine and pyrimidine nucleotide biosynthesis are shown in Table 1. Inhibitors of the essential folate synthesis with methotrexate, pemetrexed, aminopterin, raltitrexed and lometrexol, inhibitors of the committed and controlling enzyme of purine de novo biosynthesis amidophosphoribosyl-transferase (Atase) with thiopurines (azathioprine, etc), inhibitors of the guanosine synthesis by inosine monophosphate dehydrogenase (IMPDH) with mycophenolic acid, pyrazofurin. For the pyrimidine nucleotide de novo biosynthesis pathway, inhibition of dihydroorotate dehydrogenase with brequinar and leflunomide, the inhibition of the orotidine monophosphate decarboxylase with 6-azauridine, the inhibitors of key enzyme responsible for cytidine triphosphate synthesis; CTP synthase with CPEC or 3-deazauridine, inhibitors of the first committed step of pyrimidine biosynthesis aspartate carbamoyltransferase (ATCase) with phosphonoacetyl-L-aspartate (PALA). Some inhibitors of the DNNB pathway can inhibit more than one enzyme and sometimes can inhibit enzymes belonging to both purine and pyrimidine DNNB pathways. Inhibition of these different enzymes of the DNNB will potentiate the effect of antiviral nucleobases.

Embodiments of DNNBi include purine and pyrimidine analogs.

Embodiments of DNNBi include:
phosphoribosylpyrophosphate amidotransferase inhibitors 6-methylmercaptopurine riboside, 6-methylthiopurine, 6-mercaptopurine, 6-mercaptopurine riboside, 6-thioguanine, 2-amino-6-mercaptopurine riboside, azathioprine, and 2-amino-6-methylmercaptopurine;
folate synthesis inhibitors methotrexate, pemetrexed, folinic acid hDHFR, aminopterin, and trimethoprim;
1,4-naphthoquinone, lometrexol, mycophenolic acid, ribavirin, phosphonoacetyl-L-aspartate (PALA);
dihydroorotate dehydrogenase inhibitors brequinar and leflunomide;
orotidine monophosphate decarboxylase inhibitors pyrazofurin and 6-azauridine;
CTP synthase inhibitor 3-deazauridine;
glutamyltransferase inhibitor azaserine; and multitarget inhibitor 6-diazo-5-oxo-L-norleucine (DON).

### Combinations of Antiviral Nucleobases and DNNBi

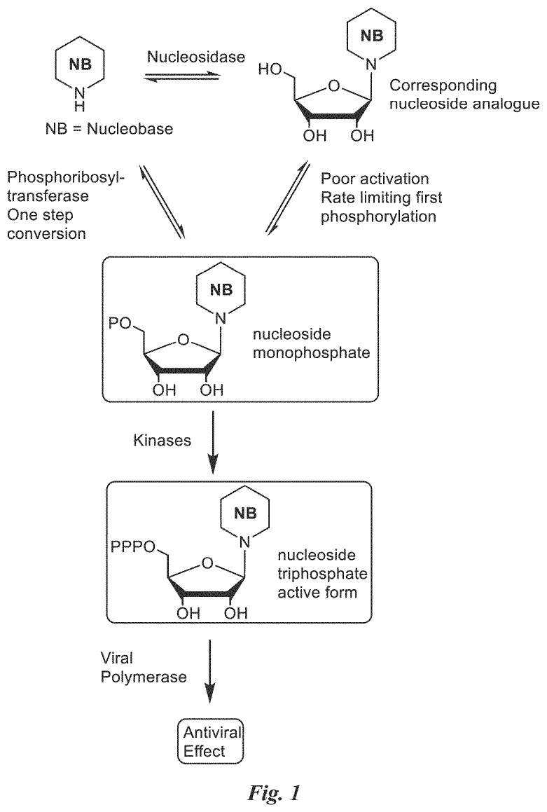

Antiviral nucleoside identification can be hindered by difficulties in chemical synthesis and poor conversion of the nucleoside to the active triphosphate form. To circumvent these issues, nucleobases were screened as antiviral agents because of their different activation pathway to the active nucleotide (FIG. 1), their low cost and ready commercial availability. Phosphoribosyl transferases of the cellular nucleotide salvage pathway directly convert some nucleobases to the corresponding nucleoside monophosphate and therefore the corresponding nucleoside analogue need not be an efficient substrate for a nucleoside kinase as shown in FIG. 1 (S. C. Sinha, and J. L. Smith, in Curr Opin Struct Biol. (England, 2001), vol. 11, pp. 733-739; L. Naesens et al (2013) Mol. Pharmacol. 84:615-629). In that regard, 3a and analogue 5a (Table 1) are substrates of human phosphoribosyl transferases and are converted in one step to the corresponding nucleoside monophosphate.

The combinations of the invention comprise: (i) a nucleobase with antiviral activity; and (ii) a potentiator compound that promotes the conversion of the antiviral nucleobase to their active forms or promotes the use of the active forms by reducing the pool of normal cellular triphosphates. Specifically, these combinations display synergistic anti-DENV, anti-ZIKV, anti-influenza and anti-coronavirus OC43 surrogate for SARS-cov-2 properties. The invention relates to chemically stable combinations of structurally diverse antiviral agent nucleobases and potentiators; inhibitors of the de novo nucleotide biosynthesis.

The combinations of the invention display synergistic anti-DENV, anti-ZIKV Anti-influenza and anti-coronavirus OC43 surrogate for SARS-cov-2 properties. The combinations of the invention increase synergistically the antiviral effect of favipiravir and similar molecules against DENV, ZIKV, influenza, and coronavirus OC43 surrogate for SARS-cov-2. The combinations have the broad antiviral properties of favipiravir. Therapeutic combinations with favipiravir are applicable to target flaviviruses (such as DENV, ZIKV, influenza, and coronavirus OC43 surrogate for SARS-cov-2), influenza viruses and other RNA viruses, including so-called “emerging viruses”. Other therapeutic combinations include antiviral nucleobases structurally similar or operating by a mechanism of action related to favipiravir. In an exemplary embodiment, the combination is synergistically active against DENV, ZIKV, influenza, and coronavirus OC43 surrogate for SARS-cov-2 and includes favipiravir, an antiviral nucleobase, and a de novo nucleotide biosynthesis inhibitor (DNNBi) such as 6-MMPR.

The use of the combinations of the invention may result in an equivalent or better antiviral effect than an antiviral compound alone and reduces the administrated dose and toxicity. Lower overall drug doses can decrease the rate of occurrence of drug-resistant variants of the targeted virus. Lower drug doses predict better patient compliance when pill burden is decreased or dosing schedule is simplified, particularly when synergy between compounds is obtained.

Nucleobase and nucleoside DENV inhibitors were screened for activity and toxicity at 10 μM and 50 μM using a luciferase-reporting DENV replicon cell line, BHK pD2-hRucPac-2ATG30 (K. Whitby et al (2005) J. Virol. 79:8698-8706). Compounds that demonstrated inhibitory activity against the replicon cell line were used in dose-response analysis to assign EC₅₀ and CC₅₀ values. The nucleobases were generally more active with a higher tissue culture therapeutic index (CC₅₀/EC₅₀) than their corresponding nucleosides (Table 1). The EC₅₀ values of the active nucleobases range from 2.4 to 110 μM, comparable to the EC₅₀ values of the active nucleosides that range from 1.3 to 113 μM (Table 1). Nucleobase 3a is 5 times more active than nucleobase 5a (favipiravir). The CC₅₀ values of the nucleobases 1a, 3a and 5a were beyond 66504 (Table 1). Nucleobase 1a did not show cytotoxicity at 1000 μM compared to 1b nucleoside (Table 1) where the CC₅₀ was 20 μM (L. Qiu, et al (2018) PloS Negl Trop Dis 12, e0006421).

TABLE 1
Active Nucleobases and Corresponding Nucleotides

| | | Name | EC₅₀ | CC₅₀ | |
|---|---|---|---|---|---|
| No. | Structure | IUPAC name | (μM) | (μM) | TTI^{b} |
| 1a | 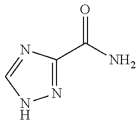 | ribavirin nucleobase 1H-1,2,4-triazole-3- carboxamide | 4.9 ± 1.6 | >1000 | 204 |
| 1b | 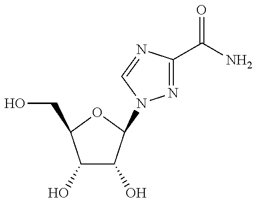 [SMILES: NC(=O)c1ncn([C@@H]2O[C@H](CO)[C@@H](O)[C@H]2O)n1] | ribavirin 1-((2R,3R,4S,5R)-3,4- dihydroxy-5- (hydroxymethyl)tetrahydro- furan-2-yl)-1H-1,2,4-triazole- 3-carboxamide CAS Reg No. 36791-04-5 | 1.3 ± 0.1 | 20 ± 0.7 | 15 |
| 2a | 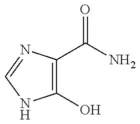 | mizoribine nucleobase 5-hydroxy-1H-imidazole-4- carboxamide | 2.4 ± 0.1 | 23 ± 4.2 | 9.6 |
| 2b | 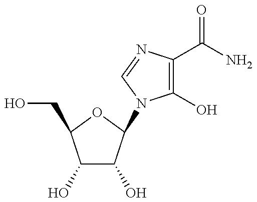 [SMILES: NC(=O)c1ncn([C@@H]2O[C@H](CO)[C@@H](O)[C@H]2O)c1O] | mizoribine 1-((2R,3R,4S,5R)-3,4- dihydroxy-5- (hydroxymethyl)tetrahydro- furan-2-yl)-5-hydroxy-1H- imidazole-4-carboxamide | 15 ± 7.8 | 33 ± 12 | 2.2 |
| 3a | 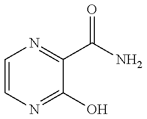 | T-1105 3-hydroxypyrazine-2- carboxamide | 21 ± 0.7 | >665 | 32 |
| 3b | 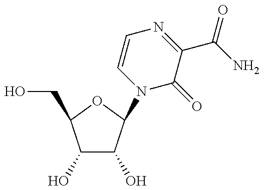 [SMILES: NC(=O)c1nccn([C@@H]2O[C@H](CO)[C@@H](O)[C@H]2O)c1=O] | T-1106 4-((2R,3R,4S,5R)-3,4- dihydroxy-5- (hydroxymethyl)tetrahydro- furran-2-yl)-3-oxo-3,4- dihydropyrazine-2- carboxamide | 113 ± 11 | >1000 | 8.8 |
| 4a | 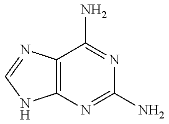 [SMILES: Nc1nc(N)c2nc[nH]c2n1] | diaminopurine 9H-purine-2,6-diamine | 3.6 ± 1.3 | 13 ± 2.1 | 3.6 |
| 4b | 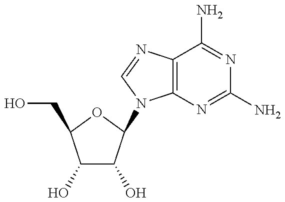 [SMILES: Nc1nc(N)c2ncn([C@@H]3O[C@H](CO)[C@@H](O)[C@H]3O)c2n1] | diaminopurine riboside (2R,3R,4S,5R)-2-(2,6- diamino-9H-purin-7-yl)-5- (hydroxymethyl)tetrahydro- furan-3,4-diol | 27 ± 3.5 | 31 ± 1.4 | 1.1 |
| 5a | 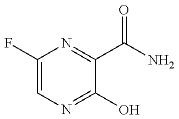 [SMILES: NC(=O)c1nc(F)cnc1O] | T-705 favipiravir 6-fluoro-3-hydroxypyrazine- 2-carboxamide | 110 ± 30 | >1000 | 9.1 |
| 6 | 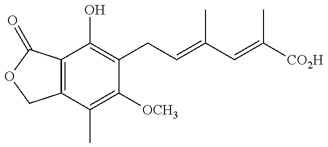 [SMILES: COc1c(C)c2c(c(O)c1C/C=C(C)/C=C(\C)C(=O)O)C(=O)OC2] | mycophenolic acid^{a} (2E,4E)-6-(4-hydroxy-6- methoxy-7-methyl-3-oxo- 1,3-dihydroisobenzofuran-5- yl)-2,4-dimethylhexa-2,4- dienoic acid CAS Reg. No. 24280-93-1 | 0.3 ± 0.03 | 1.4 ± 0.4 | 4.7 |

^{a}non-nucleobase/nucleoside control inhibitor
^{b}tissue culture therapeutic index (CC₅₀/EC50)

Inhibitors were identified using a DENV replicon BHK cell line. Inhibitory activity was verified in human cells (Huh-7) using replication-competent DENV. Dose-response experiments were conducted for 3a and 3b using a titer-reduction assay with Huh-7 cells to measure antiviral activity as previously described (S. K. Vernekar et al (2015) J. Med. Chem. 58:4016-4028). The values obtained for the compounds (Table 2) were consistent with those from the replicon assay. The replicon and Huh-7 results demonstrate that nucleobases such as 5a (favipiravir) and the related 3a display anti-DENV activity in multiple experimental systems including in human cells and with replication competent DENV virus.

TABLE 2
Titer Reduction Assay Dose-Response

| Compound No. | EC₅₀ (μM) | CC₅₀ (μM) |
|---|---|---|
| 3a | 20 ± 11 | >1000 |
| 3b | 60 ± 22 | >1000 |

Broad antiviral activity and low cost of 5a (favipiravir) are relevant properties to treat multiple viruses. However, the sub-optimal plasma concentration observed during the JIKI trial might also be a problem in trials against other viruses since Favipiravir plasma concentration is depending on the host. The present invention provides a potentiation strategy to increase the efficacy of nucleobases such as favipiravir by using a drug combination approach. A nucleobase/DNNBi combination is applicable to an antiviral approach. Inhibition of the de novo nucleotide biosynthesis pathway results in at least three possible effects relevant to uses in combination with antiviral nucleobases against viruses. First, the inhibition of the de novo nucleotide biosynthesis pathway results in the accumulation of PRPP (FIG. 2) which displaces the reversible conversion of the nucleobase by the salvage pathway toward the synthesis of the nucleoside monophosphate, consequently favoring activation of the antiviral nucleobase. Second, inhibition of the de novo nucleotide biosynthesis pathway blocks the formation of endogenous nucleotides that can compete with the antiviral nucleoside triphosphates to be used by the viral polymerase. Third, the inhibition of the de novo nucleotide biosynthesis pathway is known to trigger the innate immune response which would add another dimension to the antiviral effect of the antiviral nucleobase. The methods of the invention combine a direct-acting antiviral drug potentiated by a host cell immune defense activation. A combination of diverse antiviral approaches brought about by the combination treatment may lead to an efficacy boost and avoid drug resistance.

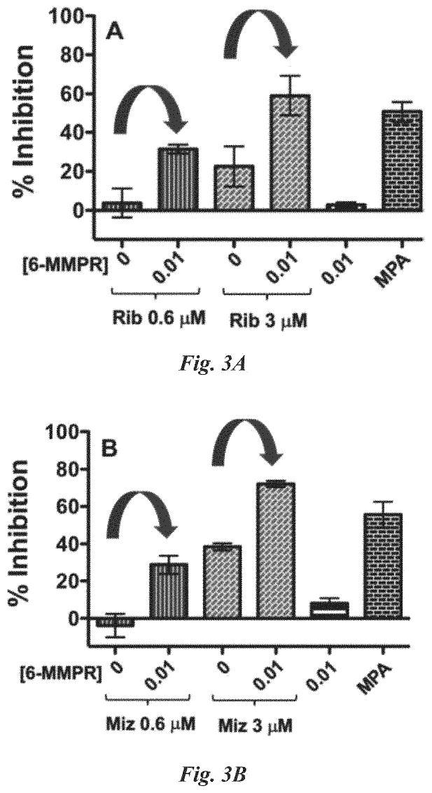

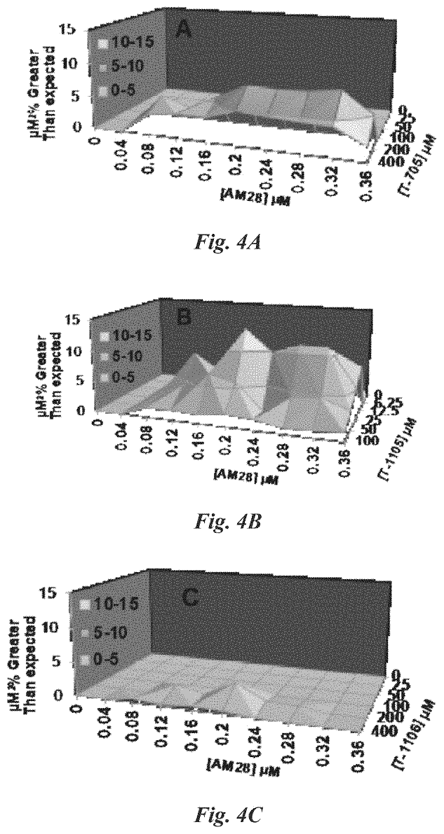

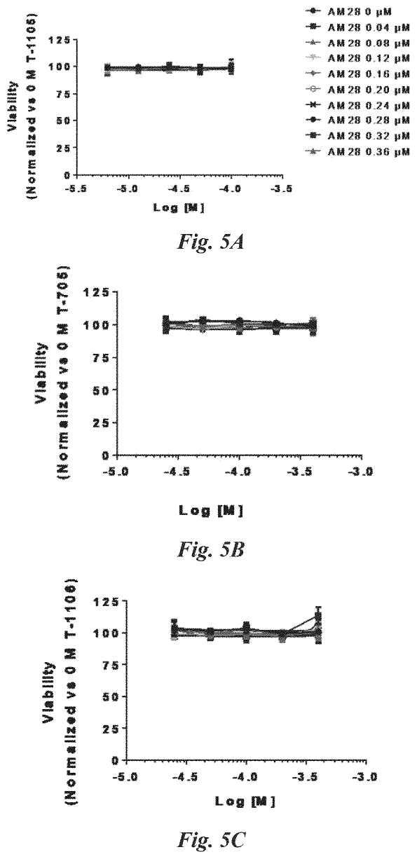

A DENV replicon system was employed to screen DNNBi for their ability to potentiate the anti-replicon activities of nucleobases. DNNB inhibitor 6-MMPR (FIGS. 3A and 3B) is a strong potentiator of nucleobase anti-replicon activities. When 1a (ribavirin nucleobase) and 2a (mizoribine nucleobase) were supplemented with 0.01 μM (micromolar) 6-MMPR an increase in antiviral activity was observed (FIGS. 3A and 3B arrows). This is an especially striking effect at the lower concentration (0.6 μM) of nucleobase where there is no antiviral activity until supplemented with 0.01 μM of 6-MMPR (FIGS. 3A and 3B, see arrows). Also striking is the lack of antiviral activity for 6-MMPR alone at 0.01 μM demonstrating that neither compound alone is effective at those specific concentrations but the combination is. There was also a 0.01 μM 6-MMPR potentiation of antiviral activity at the 3 μM nucleobase dose that already displays 20-30% inhibitory activity. Lower concentrations of 6-MMPR (0.0025 and 0.005 μM) did not potentiate nucleobase anti-replicon activity nor did they have anti-replicon activity of their own. Combinations of 6-MMPR and another nucleobase, 5a (favipiravir), were analyzed for their ability to inhibit the DENV replicon (FIG. 4A) and to determine if those effects were additive or synergistic. Combinations of ten 6-MMPR concentrations and six T-705 concentrations were added to a 96-well plate using a ten by six matrix in a standard DENV replicon assay. The results were used to investigate possible synergy or antagonism by the Bliss independence model using MacSynergy II software (Prichard, M. N. et al (1990) Antiviral Res 14:181-205; Smee, D. F. and Prichard, M. N. (2017) Antiviral Research 145:1-5) where interpretation of drug-drug interactions is based on a Volume of Synergy (VS) calculation, which can be positive (synergy), negative (antagonistic), or neutral (no or minimal interaction). The three-dimensional surface plot of the anti-DENV activities for the combinations show synergy via peaks above a plane representing additive effects and antagonism via depressions below the additive plane. The volumes of synergy across all tested combinations were within the range of 0 to +20 μM² % indicating synergy for many of the combinations (FIG. 4A). The parameter μM² % is a volume MacSynergy calculates from the 3D graph of synergy results. It uses the volume of the synergy peaks that arise out of the plane of additivity (0 μM² %) to quantitate the volume of synergy produced. The greater the volume of the synergy peak(s), the greater the synergy. Synergistic interactions appear as peaks whose heights are the percent above the calculated additivity plane. The volumes are equal to the relative amount of synergy produced per change in the two drug concentrations. Stronger synergy was observed for 3a and 6-MMPR (FIG. 4B). Antimetabolite 6-MMPR may be pre-incubated prior to nucleobase addition as a way to elicit optimal inhibition/synergy. The 6-MMPR/3b (T-1106) combination did not result in significant synergy (FIG. 4C) indicating the 6-MMPR potentiation is specific for the nucleobase form. T-1106 is a nucleoside, the synergy observed is very limited. FIG. 4C emphasizes the difference between the high synergy observed for nucleobases and potentiator (FIG. 4B) versus low synergy for corresponding nucleoside and potentiator. Cell viability was monitored in parallel to the experiments shown in FIG. 4A-C and no effect on viability was observed for any concentration or combination used as shown in FIG. 5A-C. The significance of these results is that the efficacy of nucleobase antivirals can be increased. If translatable to infected individuals, the broad but low efficacy of a nucleobase such as 5a (favipiravir) in Ebola infected patient might be able to be increased enough to enable their use as effective antivirals (D. Sissoko et al., (2016) PloS Med 13, e1001967; T. H. Nguyen et al (2017) PloS Negl Trop Dis 11, e0005389).

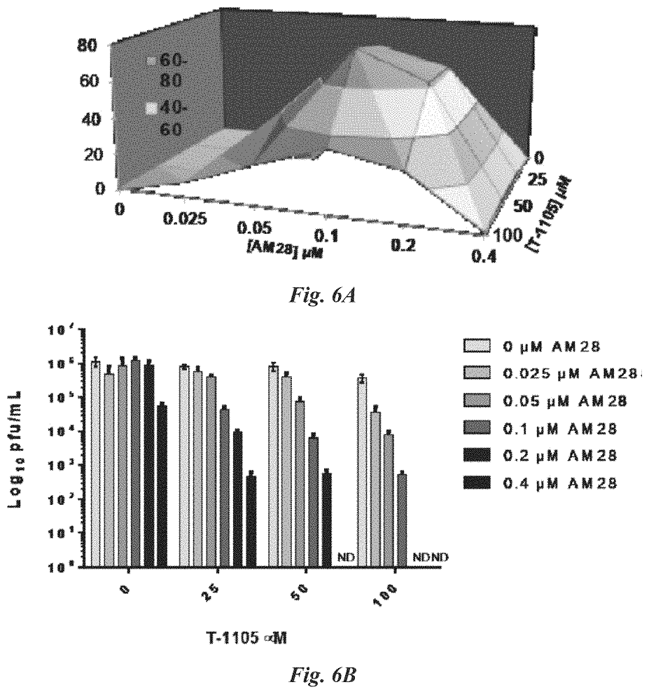

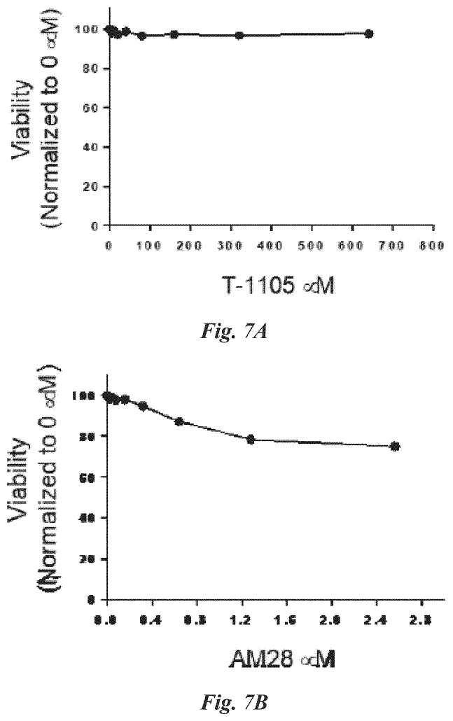

To examine nucleobase potentiation in human cells (Huh7 cells) and with replication competent virus, the drug combinations of the invention were evaluated for possible synergy in the DENV titer reduction assay. The results in FIGS. 6A and 6B show that 6-MMPR strongly potentiates the anti-DENV activity of 3a in the absence of cellular toxicity (FIGS. 7A and 7B). These results further strengthen our hypothesis that potentiation of nucleobase antiviral activity can be achieved using a DNNBi.

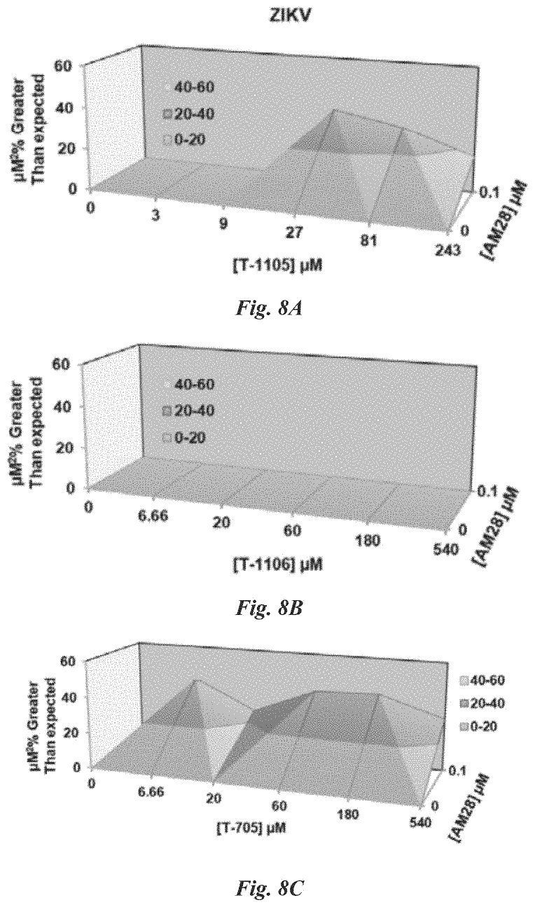

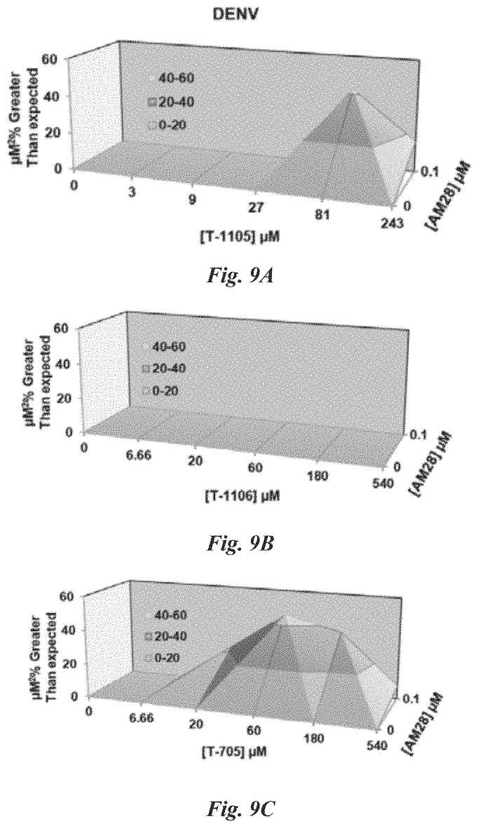

Anti-ZIKV effects of nucleobases using 6-MMPR were potentiated. Potentiation was tested using 0.1 μM (micromolar) 6-MMPR and multiple concentrations of the nucleobases 3a and 5a or the nucleoside 3b on ZIKV (FIG. 8A-C) or DENV (FIG. 9A-C) infected cells. The results for the drug combination approach in a ZIKV titer reduction assay in Huh-7 cells are shown in FIG. 8A-C. Similar to the anti-DENV results shown in FIGS. 6A-B, 7A-B, and 9A-C, inhibition of ZIKV replication with 3a or 5a (favipiravir) and 6-MMPR showed synergy and with the absence of toxicity (FIGS. 8A and 8C, respectively). Also similar to the DENV results, 6-MMPR did not potentiate the inhibition of ZIKV replication for the nucleoside 3b (see FIGS. 8B and 9B) where the combination results produced almost entirely an additive result. These results indicate the potential for an applicability of this approach to RNA viruses other than DENV.

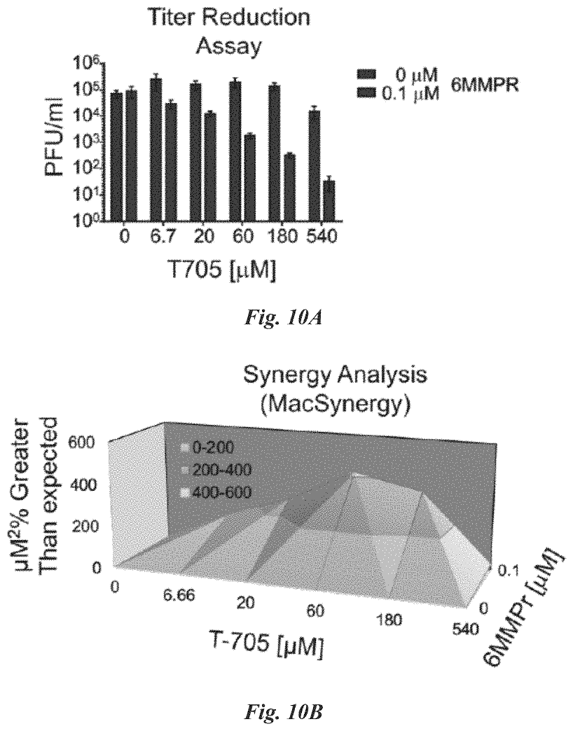

A synergistic antiviral effect of the combination of favipiravir (T-705) and 6-MMPR was demonstrated in cell models against DENV (dengue virus), Zika, HCoV-OC43 (human betacoronavirus OC43), a surrogate for SARS-CoV-2 and IAV (influenza A virus). FIG. 10A shows a plot of results of a Titer Reduction assay where 6-MMPR (6-methylmercaptopurine riboside) synergistically increases the ability of favipiravir (T705) to inhibit the production of infectious coronavirus particles. FIG. 10B shows a three-dimensional plot of synergy analysis by MacSynergy with varying concentrations of favipiravir (T705) and 6-MMPR. The dome indicates a clear synergistic effect.

At this nascent period of characterization of SARS CoV-2 infection models, HCoV-OC43 (human betacoronavirus OC43) appears to be the closest related human coronavirus to SARS 2 phylogenetically with a robust biosafetly level 2 cell culture system and thus appropriate for these studies (Vijgen L., et al (2020) J. Virol. 79(3):1595-1604; Coronaviridae Study Group of the International Committee on Taxonomy of Viruses. (2020) Nat. Microbiol. 5:536-544).

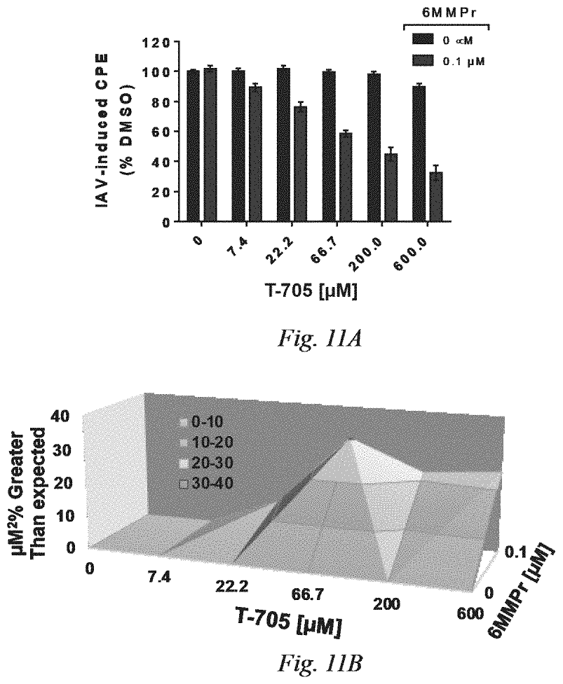

FIG. 11A shows that favipiravir (T-705) can protect cells from IAV-induced cytopathic effects (CPE) when used in combination with 6MMPR. In FIG. 11B, analysis of the CPE results (FIG. 11A) shows a clear synergy peak for the T-705/6MMPR combination.

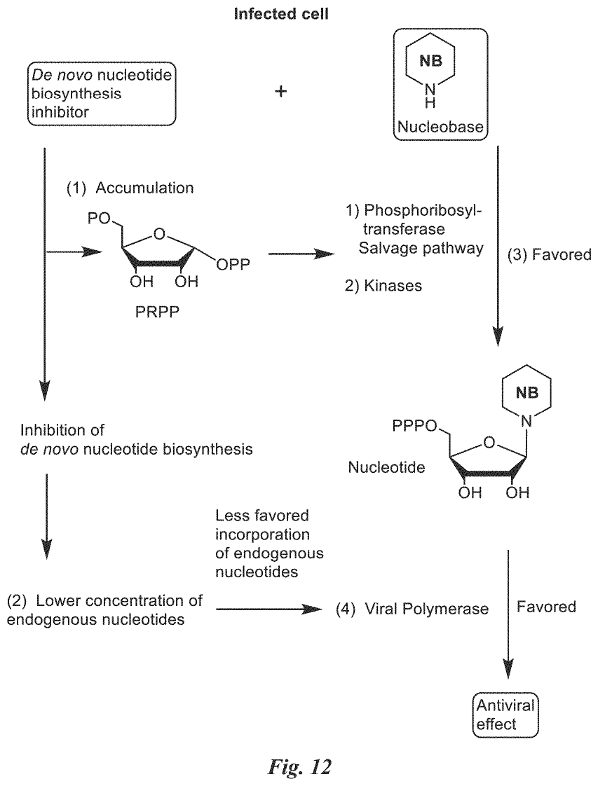

FIG. 12 shows a flow chart of the synergistic potentiation mechanism of treatment of an infected cell with antiviral-nucleobases by de novo nucleotide biosynthesis inhibitors. Inhibition of the de novo nucleotide biosynthesis results in: (i) accumulation of PRPP-phosphoribosyl pyrophosphate, and (ii) lower concentrations of endogenous nucleotides. Antiviral nucleobases benefit from the accumulation of PRPP to be converted to nucleotides and have less competition from endogenous nucleotides to be used by the viral polymerase.

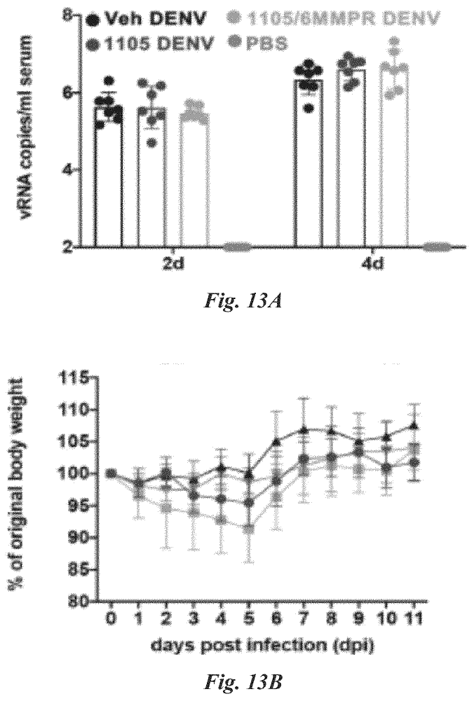

The combination of favipiravir (T705) and 6-MMPR was demonstrated to be safe in a mouse model where the combination did not impact the health of the mice beyond a critical point. FIG. 13A shows a plot of viral RNA copies per ml serum from a DENV mouse model. The mice were treated with Vehicle, the combination T-1105 (3a)+6-MMPR, 1105 only, and PBS. Viral genomes were detected and quantitated by RT-qPCR at 2 or 4 days post infection. FIG. 12B shows a plot of body weight from mice during the study of FIG. 12A where the mean plus error for percent original body weight for 11 days post-infection. These studies help predict a safe dose and timing of administration of the combination of favipiravir (T705) and 6-MMPR to treat RNA virus infections, such as SARS-CoV-2.

The potentiation strategy may not only rescue/potentiate favipiravir but also offer additional applications for favipiravir and other antiviral nucleobases against other viruses.

The experimental results described herein using DENV, ZIKV, IAV and HCoV-OC43 demonstrate the potent antiviral activities and low toxicities of certain nucleobases. In general, nucleobases were more potent and less toxic than their corresponding nucleosides and, in the case of favipiravir, the nucleobase may be more stable than its corresponding nucleoside. Synergistic antiviral effects were observed when combining a nucleobase with a DNNB inhibitor (DNNBi). The results four distinct human viral pathogens indicate that the combination approach is applicable to other viruses for increasing the efficacy, reducing cost and toxicity of antiviral nucleobases. The combination therapy methods may be efficacious against other RNA viruses with pandemic potential such as SARS-CoV, SARS-CoV-2, MERS, Ebola virus and Nipah virus.

### Pharmaceutical Compositions

Pharmaceutical compositions and formulations of the present invention include combinations of an antiviral nucleobase, a DNNBi, and one or more pharmaceutically acceptable excipient. Pharmaceutical compositions encompass both the bulk composition and individual dosage units comprised of an antiviral nucleobase, and a DNNBi described herein, along with any pharmaceutically inactive excipients, diluents, carriers, or glidants. The bulk composition and each individual dosage unit can contain fixed amounts of the aforesaid pharmaceutically active agents. The bulk composition is material that has not yet been formed into individual dosage units. An illustrative dosage unit is an oral dosage unit such as tablets, pills, capsules, and the like. Similarly, the methods of treating a patient by administering a pharmaceutical composition is also intended to encompass the administration of the bulk composition and individual dosage units.

Suitable carriers, diluents, additives, and excipients are well known to those skilled in the art and include materials such as carbohydrates, waxes, water soluble and/or swellable polymers, hydrophilic or hydrophobic materials, gelatin, oils, solvents, water and the like. Suitable carriers also include polylactic glycolic acid (PLGA) microparticles (biodegradable materials) and lipozomes. The particular carrier, diluent or excipient used will depend upon the means and purpose for which the compound of the present invention is being applied. Suitable aqueous solvents include water, ethanol, propylene glycol, polyethylene glycols, dimethylsulfoxide (DMSO), cremophor, and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament). The formulations may be prepared using conventional dissolution and mixing procedures. For example, the bulk drug substance (i.e., compound of the present invention or stabilized form of the compound (e.g., complex with a cyclodextrin derivative or other known complexation agent) is dissolved in a suitable solvent in the presence of one or more of the excipients described above. The compound of the present invention is typically formulated into pharmaceutical dosage forms to provide an easily controllable dosage of the drug and to enable patient compliance with the prescribed regimen.

Tablet excipients of a pharmaceutical formulation of the invention may include: Filler (or diluent) to increase the bulk volume of the powdered drug making up the tablet; Disintegrants to encourage the tablet to break down into small fragments, ideally individual drug particles, when it is ingested and promote the rapid dissolution and absorption of drug; Binder to ensure that granules and tablets can be formed with the required mechanical strength and hold a tablet together after it has been compressed, preventing it from breaking down into its component powders during packaging, shipping and routine handling; Glidant to improve the flowability of the powder making up the tablet during production; Lubricant to ensure that the tableting powder does not adhere to the equipment used to press the tablet during manufacture. They improve the flow of the powder mixes through the presses and minimize friction and breakage as the finished tablets are ejected from the equipment; Antiadherent with function similar to that of the glidant, reducing adhesion between the powder making up the tablet and the machine that is used to punch out the shape of the tablet during manufacture; Flavor incorporated into tablets to give them a more pleasant taste or to mask an unpleasant one, and Colorant to aid identification and patient compliance.

Tablets containing the active ingredient(s) in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

The therapeutic combinations of the invention may be administered by any route appropriate to the condition to be treated. Suitable routes include oral, parenteral (including subcutaneous, intramuscular, intravenous, intraarterial, inhalation, intradermal, intrathecal, epidural, and infusion techniques), transdermal, rectal, nasal, topical (including buccal and sublingual), vaginal, intraperitoneal, intrapulmonary and intranasal. Topical administration can also involve the use of transdermal administration such as transdermal patches or iontophoresis devices. Formulation of drugs is discussed in Remington's Pharmaceutical Sciences, 18.sup.th Ed., (1995) Mack Publishing Co., Easton, Pa.; and Liberman, H. A. and Lachman, L., Eds., Pharmaceutical Dosage Forms, Marcel Decker, Vol 3, 2.sup.nd Ed., New York, N.Y. Where the compound is administered orally, it may be formulated as a pill, capsule, tablet, etc. with a pharmaceutically acceptable carrier, glidant, or excipient. Where the compound is administered parenterally, it may be formulated with a pharmaceutically acceptable parenteral vehicle or diluent, and in a unit dosage injectable form, as detailed below.

A dose to treat human patients may range from about 1 mg to about 2000 mg of antiviral nucleobase compound, and about 1 mg to about 2000 mg of DNNBi. A dose may be administered once a day (QD), twice per day (BID), or more frequently, depending on the pharmacokinetic (PK) and pharmacodynamic (PD) properties, including absorption, distribution, metabolism, and excretion of the particular compound. In addition, toxicity factors may influence the dosage and administration dosing regimen. When administered orally, the pill, capsule, or tablet may be ingested twice daily, daily or less frequently such as weekly or once every two or three weeks for a specified period of time. The regimen may be repeated for a number of cycles of therapy. Although treating as early as possible in the course of infection is generally accepted as most efficient, the potentiation strategy of the invention by increasing drug bioactivation might allow a delayed treatment and still provide a benefit.

### Methods of Treatment of Viral Infection

The combination compounds of the invention may be used to treat human diseases caused by RNA viruses including Ebola virus disease, severe acute respiratory syndrome (SARS), rabies, common cold, influenza, hepatitis C, West Nile fever, polio, measles. severe acute respiratory syndrome (SARS-CoV) coronavirus, severe acute respiratory syndrome 2 (SARS-CoV-2) coronavirus, Middle East respiratory syndrome (MERS) coronavirus, rabies virus, common cold viruses, IAV, hepatitis C, respiratory syncytial virus, Nipah virus, Lassa fever virus.

Patients treated with the combination of an antiviral nucleobase and a DNNBi harbor an RNA virus infection or are at risk of such an infection.

The methods of the invention include: methods of diagnosis based on the identification of a biomarker associated with an RNA virus; methods of determining whether a patient will respond to a combination of an antiviral nucleobase and a DNNBi; methods of optimizing therapeutic efficacy by monitoring clearance of the antiviral nucleobase, or the DNNBi; methods of optimizing a therapeutic regimen of an antiviral nucleobase and a DNNBi, by monitoring the development of therapeutic resistance mutations; and methods for identifying which patients will most benefit from treatment with an antiviral nucleobase and a DNNBi and monitoring patients for their sensitivity and responsiveness to treatment with the combination of an antiviral nucleobase and a DNNBi.

DENV/ZIKV are diagnosed by assaying for levels of a viral protein, NS1, or viral RNA or the presence of viral antibodies up to 7 days after onset of symptoms for individuals known to have been in areas where DENV/ZIKV infections are prevalent. IAV is diagnosed by identification of viral protein present in patient samples or viral RNA present in patient samples.

The nucleobase library examined for antiviral activity and potentiation is continuously being expanded to include previously described nucleobases, nucleobases derived from known nucleoside analogues and novel nucleobases whose structures and activities have not been disclosed. Similarly, known and novel DNNBi compounds continue to be screened for potentiation activity. Antiviral screening has also been expanded to IAV to document how nucleobase/DNNBi combinations affect IAV replication in cell culture compared to the compounds singly and approved IAV drugs. Other ongoing studies include the evaluation of nucleobase potentiation using an in vivo AG129 interferon-deficient mouse model for DENV and ZIKV infection. Current combinations including 5a or 3a with 6-MMPR will be evaluated for efficacy and side effects compared to the compounds singly and known antiviral controls. In addition, dose information obtained in this model will inform predictions of doses used to evaluate potentiation in possible future human trials.

### EXAMPLES

### Example 1 Combination Compounds

5a (T-705) (CAS #259793-96-9, 6-fluoro-3-hydroxypyrazine-2-carboxamide) was purchased from ASTA Tech. 3a (T-1105) (CAS Reg. No. 55321-99-8, 3-Hydroxy-2-pyrazinecarboxamide) was purchased from Alfa Aesar. 3b (T-1106) was synthesized according to known procedures (CA2600359). 1a (ribavirin base) (CAS Reg. No. 3641-08-5, 1,2,4-Triazole-3-carboxamide) was purchased from Ark Pharm. 1b (ribavirin) (CAS Reg. No. 36791-04-5) was purchased from Carbosynth. 2a (mizoribine base) (CAS Reg. No. 56973-26-3, 5-Hydroxy-1H-imidazole-4-carboxamide) was purchased from Ark Pharm. 2b (mizoribine) (CAS Reg. No. 50924-49-7) was purchased from Carbosynth. 4a (diaminopurine) (CAS Reg. No. 1904-98-9, 2,6-diaminopurine) was purchased from Sigma-Aldrich. 4b (diaminopurine riboside) (CAS Reg. No. 2096-10-8, 2-Aminoadenosine) was purchased from Berry and Associates. 6 (mycophenolic acid) (CAS Reg. No. 24280-93-1) was purchased from Sigma-Aldrich. 6-Methyl-mercaptopurine riboside (6-MMPR, CAS Reg. No. 324-69-8) named as (2R,3S,4R,5R)-2-(hydroxymethyl)-5-(6-(methylthio)-9H-purin-9-yl)tetrahydrofuran-3,4-diol, was purchased from Sigma-Aldrich and has the structure:

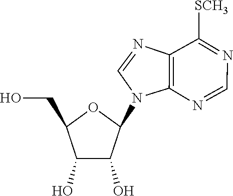 [SMILES: CSc1ncnc2c1ncn2[C@@H]1O[C@H](CO)[C@@H](O)[C@H]1O]

### Example 2 Cell Lines and Virus

The hepatocyte-derived cellular carcinoma cell line Huh-7 was used for DENV infection and drug treatment (H. Nakabayashi, et al (1982) Cancer Res 42:3858-3863). The African green monkey kidney Vero cell line (ATCC CRL-81) was used to titer DENV via plaque assay. The baby hamster kidney cell line carrying a DENV subgenomic replicon, BHK pD2-hRucPac-2ATG30 (K. Whitby et al (2005) J. Virol. 79:8698-8706), obtained from Dr. M. Diamond, Washington University, School of Medicine), was used for DENV replicon assay. All cell lines were maintained in Dulbecco's modified Eagle's (DME) medium supplemented with 10% fetal bovine serum (FBS), 100 IU streptomycin/penicillin per ml and 10 μg/mL plasmocin (InvivoGen) at 37° C. in a 5% CO₂ incubator. DENV replicon cells were supplemented with 3 μg/mL puromycin (Life Technologies). DENV-2 stocks from New Guinea C strain (ATCC VR-1584) and ZIKV H/PAN/2015 (ATCC NR-50219) were generated from C6/36 mosquito cell cultures (ATCC CRL-1660) grown in Minimum Essential Medium (MEM) supplemented with 10% FBS, 1% non-essential amino acids and 1% sodium pyruvate at 28° C. with 5% CO₂. The C6/36 cells on T-150 flasks were inoculated with virus and the supernatant harvested after complete cytopathic effects. Viral stock titers were determined by plaque assay on Vero cells.

### Example 3 Cell Viability Assay

The sensitivity of the cell lines to the compounds was examined using the 3-(4,5-dimethylthiazol-2-yl)-5-(3-carboxymethoxyphenyl)-2-(4-sulfophenyl)-2H-tetrazolium (MTS)-based tetrazolium reduction CellTiter 96 Aqueous Non-Radioactive cell proliferation assay (Promega G5430). The compounds were initially tested at 10 and 50 μM final concentrations. Each plate also contained DMSO alone, medium alone, and an inhibitory compound, mycophenolic acid 6 (Table 1). DENV replicon or Huh-7 cells were plated at a density of 1,500 or 8×10³ cells, respectively, per well in 96-well plates containing 100 μl of culture medium overnight. Compounds were added to triplicate wells in culture medium and incubated for an additional 72 h. MTS reagent was then added to each well and incubated at 37° C. in a humidified 5% CO₂ atmosphere. The plates were read at various time points at a wavelength of 490 nm using a Molecular Devices M5e plate reader. Mean values of triplicate wells were determined and compared to the mean value for the wells that received DMSO alone. For compounds selected for dose-response experiments, the CC₅₀ was determined by comparing cell viability for eight serial dilutions of the compound and DMSO treated cells using GraphPad Prism software. The CC₅₀ value was defined as the compound concentration resulting in a 50% reduction readout compared with the DMSO.

### Example 4 DENV Replicon Assay

Compounds were evaluated for antiviral properties using BHK cells containing a DENV-2 viral replicon. 1.5×10³ replicon-containing cells per well were plated in white opaque 96-well plates in the absence of antibiotic selection and the next day, compounds dissolved in DMSO were added to triplicate wells in culture medium. The compounds were initially tested at 10 and 50 μM final concentrations and each plate also contained DMSO alone, medium alone, and mycophenolic acid 6 (Table 1). Three days later, medium was replaced with a 1:1000 dilution of ViVi-Ren Live Cell Substrate (Promega) in DME minus phenol red and 10% FBS. Luminescence was measured with a Molecular Devices M5e plate reader. Mean values of triplicate wells were determined and compared to the mean value for the wells that received DMSO alone. For compounds selected for dose response experiments, the concentration of compound that reduced luciferase activity by 50% was defined as the 50% effective concentration (EC₅₀). The EC₅₀ was determined by comparing luciferase activity for eight serial dilutions of the compound and DMSO treated cells using GraphPad Prism software

### Example 5 Titer Reduction Assay

Huh-7 cells were seeded in 12-well plates at a density of 4×10⁵ cells per well in 1 mL culture medium. The next day, cells were washed and inoculated with DENV at a multiplicity of infection (MOI) of 0.2 in 500 μl infection medium (MEM containing 2% FBS and 10 mM HEPES). The inoculum was removed after 1 h, cells were washed with PBS and then incubated in 1 mL MEM, 2% FBS, 1% pen/strep plus compound(s) for 72 h. Viral supernatants were clarified by centrifugation for 5 min at 1500×g and aliquoted and stored at −80° C. Viral titers were determined using a plaque assay on Vero cells. Briefly, confluent Vero cell monolayers in 24-well plates were incubated at 37° C. for 1 h with duplicate 300 μl samples of 10-fold serial dilutions of viral supernatants. The cells were then washed to remove unbound viral particles and overlaid with 500 μl MEM containing 1.3% methylcellulose, 5% FBS and 10 mM HEPES. After 5 days of incubation at 37° C. and 5% CO₂, cells were washed with PBS, fixed, and stained using 1% Giemsa. Infectious virus titer (pfu/mL) was determined using the following formula: number of plaques×dilution factor×(1/inoculation volume). The viral titer was presented as the mean of duplicate samples from a dilution yielding approximately 20-50 plaques per well.

### Example 6 Determination of Potentiation Using a DENV Replicon-Containing Cell Line

DENV luciferase replicon cells 1.7×103 cells per well were plated in an opaque 96-well plate. Twenty-four hours later, columns 2 to 11 were treated with increasing concentrations of 6-MMPR (0, 0.04, 0.08, 0.12, 0.16, 0.2, 0.24, 0.28, 0.32 and 0.36 μM). Rows B to H were treated either with T-1105 (0, 6.25, 12.5, 25, 50 and 100 μM), T-705 (0, 25, 50, 100, 200 and 400 μM) or T-1106 (same concentrations as used for T-705). After 72 hours of treatment, luciferase signal was analyzed using the ViVi-ren Live Cell Substrate (Promega) diluted in DME minus phenol red and 10% FBS. Luminescence was measured with a Molecular Devices M5e plate reader. Mean values of four biological replicates were determined and expressed as percentage normalized vs DMSO control (0 μM for each drug). Synergy was determined using the MacSynergy II software. In parallel cell viability was evaluated using as mentioned above.

### Example 7 Determination of Potentiation in a Replication-Competent Virus Assay Using Huh-7 Cells

DENV and ZIKV. Huh7 cells were plated in each well of a 24 well plate (1×10⁵/well). Twenty-four hours later, cells were inoculated with DENV 2 or ZIKV at a multiplicity of infection (MOI) of 0.05. Two hours post-inoculation, inoculum was retired and fresh medium containing treatment was added. Columns 1 to 6 were treated with increasing concentrations of 6-MMPR (0, 0.025, 0.05, 0.1, 0.2 and 0.4 μM). Rows A to D were treated with 0, 25, 50 and 100 μM of T-1105. Supernatants were collected after 72 hours post-infection and infectious virions were analyzed by plaque assay as extent of infectious virus production. For FIG. 5, Huh7 cells were inoculated with DENV or ZIKV and treated with different concentrations of T-1105 (0, 3, 9, 27, 51 or 243 μM), T-1106 (0, 6.7, 20, 60, 180 or 540 μM) and T-705 (same concentrations as T-1106) in combination with 0.1 μM AM28 or DMSO. After 72 hours post infection supernatants were collected and analyzed for viral yield by plaque assay.

HCoV-OC43. Huh7 cells were 12,500 Huh7 cells were seeded in a 96 well plate. Next day, the cells were inoculated with OC43 virus at a MOI (multiplicity of infection) of 0.5 for two hours. Next, the inoculum was removed and cells were treated with different combinations of 6MMPr and T-705 or remdesivir 1 μM (RDV) as positive control. Three wells per treatment were used. Cells were incubated for 5 days. Infected and treated cells were analyzed for cell viability by MTS and absorbance was measured at 490 nm.

IAV. Huh7 cells were plated in 96 well-plates (2×10⁴ cells per well). Next day the cells were washed twice with PBS and inoculated at an MOI of 0.03 in infection medium (DMEM supplemented with 0.2% BSA, 0.5 mg/mL of TPCK-treated trypsin, 1× pen-strep, 1× glutamax, 10 mM hepes) for 2 hours. Next, the inoculum was removed and cells were treated with different combinations of 6MMPr and T-705. After 48 hours post-infection, cell viability was assayed using MTS to analyze CPE induced by IAV and the protection promoted by the treatments. Antiviral effects expressed as % of Inhibition. Absorbance values for each condition were normalized to DMSO-treated and infected cells (0% inhibition) and to non-infected cells (100% inhibition).

Although the foregoing invention has been described in some detail by way of illustration and example for purposes of clarity of understanding, the descriptions and examples should not be construed as limiting the scope of the invention. Accordingly, all suitable modifications and equivalents may be considered to fall within the scope of the invention as defined by the claims that follow. The disclosures of all patent and scientific literature cited herein are expressly incorporated in their entirety by reference.

1. A method for the treatment of an RNA virus infection comprising administering a therapeutic combination as a combined formulation or by alternation to a patient, wherein the therapeutic combination comprises therapeutically effective amounts of (i) an antiviral nucleobase or a pharmaceutically acceptable salt thereof; and (ii) a de novo nucleotide biosynthesis inhibitor (DNNBi) or a pharmaceutically acceptable salt thereof.

2. The method of claim 1 wherein the RNA virus infection is selected from dengue virus (DENV), Zika virus (ZIKV), Ebola virus, West Nile virus. severe acute respiratory syndrome (SARS) virus, Middle East Respiratory syndrome (MERS) coronavirus, rabies virus, common cold viruses, influenza, hepatitis C, West Nile fever, polio. measles, respiratory syncytial virus, Nipah virus, Lassa fever virus, and SARS-CoV-2.

3. The method of claim 1 wherein the RNA virus infection is dengue virus (DENV).

4. The method of claim 1 wherein the RNA virus infection is Zika virus (ZIKV).

5. The method of claim 1 wherein the RNA virus infection is influenza A.

6. The method of claim 1 wherein the RNA virus infection is SARS-CoV-2.

7. The method of claim 1 wherein the antiviral nucleobase is selected from the structures: 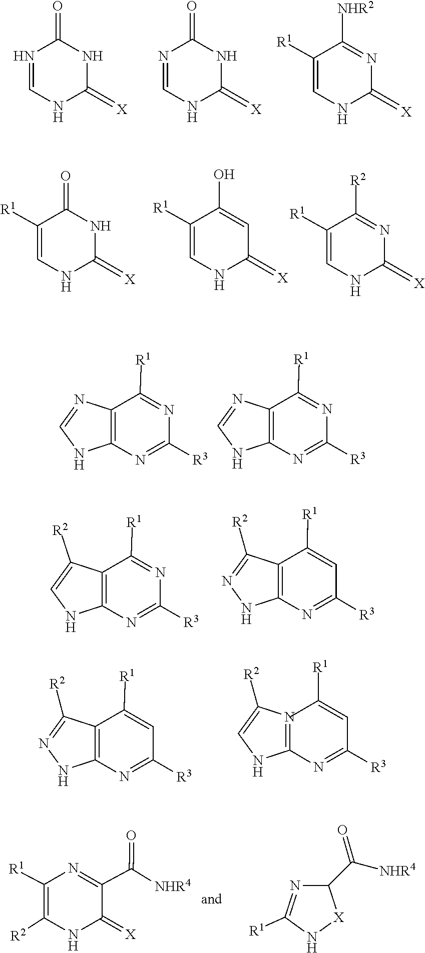
wherein:
R¹ is selected from the group consisting of H, Me, F, Cl, Br, I, OH, NH₂, SH, OMe, NO₂, NHOH, NHOMe, NHNH₂, C═ONH₂, C₁-C₈ alkyl, and 5- or 6-membered heteroaryl;
R² is selected from the group consisting of H, OH, OMe, NH₂, NHMe, C═ONH₂, C₁-C₈ alkyl, and 5- or 6-membered heteroaryl;
R³ is selected from the group consisting of H, F, Cl, Br, I, OH, S, NH₂, SH, OMe, NO₂, NHOH, NHOMe, NHNH₂, C═ONH₂, C₁-C₈ alkyl, and 5- or 6-membered heteroaryl;
R⁴ is selected from the group consisting of H, NH₂ and C₁-C₈ alkyl; and
X is NR², O or S.

8. The method of claim 1 wherein the antiviral nucleobase is selected from 1H-1,2,4-triazole-3-carboxamide, 5-hydroxy-1H-imidazole-4-carboxamide, 3-hydroxypyrazine-2-carboxamide, 9H-purine-2,6-diamine; and 6-fluoro-3-hydroxypyrazine-2-carboxamide (favipiravir).

9. The method of claim 1 wherein the DNNBi is selected from:
phosphoribosylpyrophosphate amidotransferase inhibitors 6-methylmercaptopurine riboside, 6-methylthiopurine, 6-mercaptopurine, 6-mercaptopurine riboside, 6-thioguanine, 2-amino-6-mercaptopurine riboside, azathioprine, and 2-amino-6-methylmercaptopurine;
folate synthesis inhibitors methotrexate, pemetrexed, folinic acid hDHFR, aminopterin, and trimethoprim;
1,4-naphthoquinone, lometrexol, mycophenolic acid, ribavirin, phosphonoacetyl-L-aspartate (PALA);
dihydroorotate dehydrogenase inhibitors brequinar and leflunomide;
orotidine monophosphate decarboxylase inhibitors pyrazofurin and 6-azauridine;
CTP synthase inhibitor 3-deazauridine;
glutamyltransferase inhibitor azaserine; and
multitarget inhibitor 6-diazo-5-oxo-L-norleucine (DON).

10. The method of claim 1 wherein the DNNBi is (2R,3S,4R,5R)-2-(hydroxymethyl)-5-(6-(methylthio)-9H-purin-9-yl)tetrahydrofuran-3,4-diol (6-MMPR).

11. The method of claim 1 wherein the DNNBi is a pro-drug of 6-MMPR.

12. The method of claim 1 wherein the antiviral nucleobase is favipiravir or 3-hydroxypyrazine-2-carboxamide (T-1105), and the DNNBi is 6-MMPR.

13. (canceled)

14. The method of claim 12 wherein the RNA virus infection is SARS-CoV-2.

15. The method of claim 1 wherein the therapeutic combination is administered to the patient as a combined formulation as a solid, oral dosage form in a tablet or capsule.

16. (canceled)

17. (canceled)

18. The method of claim 1 wherein the therapeutic combination is administered to the patient by alternation during a dosing regimen.

19. A method of selecting for patients likely to respond to a combination of an antiviral nucleobase and a de novo nucleotide biosynthesis inhibitor (DNNBi), wherein the patient has been previously treated with a viral RNA polymerase inhibitor.

20. The method of claim 19 wherein a biological sample from the patient has been characterized to contain an NS1 viral protein, viral RNA or the presence of viral antibodies within 7 days after onset of symptoms associated with an RNA viral infection.

21. The method of claim 2 wherein replication of the virus is inhibited.

22. A pharmaceutical composition comprising therapeutically effective amounts of: (i) an antiviral nucleobase, (ii) a de novo nucleotide biosynthesis inhibitor (DNNBi), and (iii) an excipient in solid, oral dosage form as a tablet or capsule.

23. (canceled)

24. (canceled)

25. The pharmaceutical composition of claim 22 wherein the antiviral nucleobase and (DNNBi) are in synergistic amounts.

26. The pharmaceutical composition of claim 25 wherein the antiviral nucleobase is favipiravir or 3-hydroxypyrazine-2-carboxamide (T-1105), and the DNNBi is 6-MMPR.

27. (canceled)
